(19) United States
(12) Patent Application Publication
(10) Pub. No.: US 2026/0146299 A1
(43) Pub. Date: May 28, 2026

(54) Electrochemical Genosensor Based on Target-Triggered DNAzyme Activity

(71) Applicant: The Penn State Research Foundation, University Park, PA (US)

(72) Inventors: Dipanjan Pan, University Park, PA (US); Maha Mohammad Shehadeh Alafeef, University Park, PA (US)

(73) Assignee: The Penn State Research Foundation, University Park, PA (US)

(21) Appl. No.: 19/377,720

(22) Filed: Nov. 3, 2025

Related U.S. Application Data
(60) Provisional application No. 63/715,780, filed on Nov. 4, 2024.

(51) Int. Cl. C12Q 1/70; C12Q 1/00; C12Q 1/28; C12Q 1/6806; C12Q 1/6844

(52) U.S. Cl. CPC C12Q 1/708; C12Q 1/005; C12Q 1/28; C12Q 1/6806; C12Q 1/6844; G01N 2333/908

(57) ABSTRACT

Embodiments relate to methods and processes for screening and detecting infectious diseases. Embodiments further relate to a molecularly driven (nucleic acid) method and process for screening and detecting infectious diseases using an electrochemical genosensor with a highly specific cleavage-triggered DNA polymerization reaction.

### CROSS-REFERENCE TO RELATED APPLICATIONS

This patent application is related to and claims the benefit of priority of U.S. Provisional Application 63/715,780, filed on Nov. 4, 2024. The entire contents of this application is incorporated by reference.

### INCORPORATION BY REFERENCE STATEMENT REGARDING SEQUENCE LISTINGS

The content of the XML file of the ST.26 SEQUENCE LISTING named “0073605-000705.xml”, which is 10,027 bytes in size, was created on Oct. 31, 2023 and is electronically submitted herewith via Patent Center, and is hereby incorporated by reference in its entirety.

### FIELD OF THE INVENTION

Embodiments relate to methods and processes for screening and detecting infectious diseases. Embodiments further relate to molecularly driven (nucleic acid) methods and processes for screening and detecting infectious diseases using an electrochemical genosensor.

### BACKGROUND OF THE INVENTION

Globally, cervical cancer is the fourth most common cancer prevailing in women and the seventh most common cancer in terms of overall cancer incidences. 80% of the deaths from cervical cancer occur in regions lacking adequate screening infrastructures or ready access to such screening infrastructures. In 2019, an estimated 283.15 million women were diagnosed with cervical cancer worldwide, and about 311,000 women died from the disease in 2018.

Cervical cancer (or pre-cancer) is preventable and/or treatable if and only if it is diagnosed early. Despite the acceptance of cytologic testing as the primary screening method for cervical cancer, cytologic testing has shown a high false negative rate. Studies have shown that 20% to 40% of new cervical cancer cases are diagnosed in women who have had “proper” screening, where the diagnostic sensitivity of the Pap smear was found to be 51%. Furthermore, cytological testing is still complex and expensive in high-income countries and may not be feasible in low-income countries.

Sexually transmitted infections caused by strains of the human papillomavirus (HPV) have been found to play a role in causing most cervical cancer cases. Early-stage cervical cancer generally produces no signs or symptoms, and it can take many years for an HPV infection to develop into cancer. Different HPV types can be classified into high risk (HR) and low risk (LR) categories. The HR-HPV is shown to be associated with pre-neoplastic lesions and carcinomas and include types 16, 18, 31, 33, 35, 39, 45, 51, 52, 56, 58, 59, 68. On the other hand, LR-HPV is associated with wart formation and include types 6, 11, 40, 42, 43, 44, 54, 61, 70, 72, 81. Due to the limitations of the Pap smear and an improved understanding of the role of HPV in cervical carcinogenesis, primary prevention has shifted to HR-HPV testing. The American Society for Colposcopy and Cervical Pathology has recommended the use of HR-HPV testing in a variety of situations. Additionally, the World Health Organization (WHO) is calling for twice-in-a-lifetime testing as a part of the Global Cervical Cancer Elimination strategy. However, this goal may not be feasible using currently available HPV tests.

### SUMMARY

We determined that there is a need to develop a point-of-care test to screen for HPV that can be operated outside the traditional healthcare setting and accessed by areas of low income and limited resources. We also determined that there is an unmet need for new methods and processes that can enable fast, reliable, and scalable detection of target nucleic acids of interest, such as ribonucleic acid (RNA) and deoxyribonucleic acid (DNA), for infectious disease (e.g., HPV) diagnosis.

Several methods of RNA and DNA detection have been previously developed. For example, quantitative polymerase chain reaction (qPCR) technology has been developed to amplify different regions of viral genomes using a variety of primer combinations. Although qPCR technology is an effective and accurate tool for detecting and genotyping viruses, it is a complex, time and labor-intensive process that requires expert personnel and specialized infrastructure. Additionally, thermal cycling of the qPCR technique imposes instrumental constraints, limiting the technique to a laboratory setting, and dual-labelled fluorescent probes, such as Taqman probes, are usually needed to determine the specificity of amplification.

Therefore, isothermal amplification of RNA, such as nucleic acid sequence-based amplification, rolling-cycle amplification, and loop-mediated isothermal amplification have emerged as alternative amplification techniques. As the above-mentioned techniques can proceed at a constant temperature, there is no need for specialized instruments for RNA detection, and in addition, they have potential for “on-site” and point-of-care testing.

Although isothermal amplification-based testing techniques, such as colorimetric RT-LAMP, provide sensitive and accurate results, the method still suffers from a high incidence of false-positive results due to the presence of spurious amplification byproducts.

Moreover, with respect to HPV, the genotyping of HR-HPV is necessary to evaluate a subject case to allow for early intervention and to avoid the development of cervical cancer. Even though the detection and genotyping of all individual HPV types in a sample is not necessary, it is nevertheless important to detect all HR-HPVs. However, only one type of HPV can be detected in a single isothermal amplification reaction, making the test labor intensive, time consuming, and ultimately unsuitable for HPV genotyping in specimens containing multiple HPV types.

Ultimately, despite these advancements in diagnostics, on-site and point-of-care testing requires new processes and methods to help better address the above noted short comings in more conventional diagnostic approaches.

Embodiments therefore relate to apparatuses, methods and processes for screening and detecting infectious diseases (e.g., HPV). Embodiments further relate to a molecularly driven (nucleic acid) method and process for screening and detecting infectious diseases using an electrochemical genosensor with a highly specific cleavage-triggered DNA polymerization reaction. Some embodiments can be configured for utilization of a mobile reader (e.g., a handheld reader). For instance, the use of a hand-held reader, especially a portable reader, can allow for testing to be scaled-up, can enable testing efforts to be established globally, and can help speed up the commercialization potential.

In an exemplary embodiment, a method for detecting an infectious disease can include collecting a sample from a subject, wherein the sample comprises RNA and/or DNA of the subject; introducing the sample to a reagent solution to form a reaction mixture, wherein the reagent solution comprises at least one DNAzyme, wherein the DNAzyme is designed to target and amplify an RNA and/or DNA expression profile linked to an infectious disease; and adding an oxidizing solution to the reaction mixture to form a detection mixture, wherein the oxidizing solution comprises at least one organic substrate and hydrogen peroxide. The oxidizing solution can facilitate an oxidation reaction that, if detected, can indicate the sample has the infectious disease. If the oxidation reaction is not detected (e.g., it does not occur or does not occur with a sufficient intensity to meet a pre-selected detection threshold), the sample can be found to not have the infectious disease.

In some embodiments, the method can include mixing the sample, the reagent solution, and the oxidizing solution at the same time. In other embodiments, the sample can first be mixed with the reagent solution to form a reaction mixture. The reaction mixture can then be mixed with the oxidizing solution to form a detection mixture for detection of the oxidation reaction.

Embodiments of the method can be implemented via an apparatus. The apparatus can include a housing that can receive the sample, oxidizing solution, and reagent solution for forming a detection mixture for depositing on a detector element (e.g., a detection strip). The detector element can be a sensor or other type of detector for detecting the presence of the oxidation reaction to determine whether the infectious disease is present in the sample. The detector can be evaluated via a reader. The reader can collect the data and either analyze and output a detection response from the data or communicate the data to at least one external device (e.g., a computer device) for storage and analysis of the data to determine whether the sample has the infection disease. In some configurations, the external device can include a smart phone, tablet, laptop computer, or personal computer or other type of communication terminal. In some implementations, the external device can utilize an application supported by a remote server (e.g., cloud-based server, server connectable via an internet connection or network connection, etc.) to facilitate data analysis of the reader's data and providing an output on whether the sample has the infectious disease or not.

In some embodiments, the reaction mixture comprises a horseradish peroxidase-mimicking DNAzyme, and the horseradish peroxidase-mimicking DNAzyme is configured to catalyze an oxidation reaction of the at least one organic substrate.

In some embodiments, the infectious disease to be detected is selected from the group consisting of SARS-CoV-2, HPV, HCV, syphilis, chlamydia, and gonorrhea.

In some embodiments, the oxidizing solution further comprises hemin.

In some embodiments, a sample can be treated with a buffer configured to stabilize the RNA and/or DNA of the subject prior to introducing the sample to the reagent solution.

In some embodiments, the at least one organic substrate can be selected from the group consisting of ABTS, OPD, AmplexRed, DAB, AEC, TMB, homovanillic acid, luminol, and mixtures thereof.

Other details, objects, and advantages of our process for screening and detecting infectious diseases, apparatuses for screening and detecting infectious diseases, systems for screening and detecting infectious diseases, and methods of making and using the same will become apparent as the following description of certain exemplary embodiments thereof proceeds.

### BRIEF DESCRIPTION OF THE DRAWINGS

The above and other objects, aspects, features, advantages, and possible applications of embodiments of the present innovation will be more apparent from the following more particular description thereof, presented in conjunction with the following drawings. Like reference numbers used in the drawings may identify like components.

FIG. 1 shows an exemplary method and process for screening and detecting infectious diseases.

FIG. 2 is a table of DNAzyme sequences designed for targeting SARS-CoV-2.

FIG. 3 is a table of DNAzyme sequences designed for targeting HR-HPV.

FIG. 4 show an exemplary electrochemical genosensor.

FIG. 5 shows an exemplary smartphone-based electrochemical genosensor and an exemplary method and process of using thereof.

FIG. 6 shows optimization of DNAzyme sequences designed for targeting SARS-CoV-2 using cyclic voltammetry (CV).

FIG. 7 shows optimization of DNAzyme sequences designed for targeting SARS-CoV-2 using differential pulse voltammetry (DPV).

FIG. 8 shows optimization of DNAzyme sequences designed for targeting SARS-CoV-2 using square wave cyclic voltammetry (SWV).

FIG. 9 shows time optimization of reaction conditions.

FIG. 10 shows optimization of DNAzyme sequences designed for targeting HR-HPV using CV.

FIG. 11 is a block diagram illustrating an exemplary embodiment of a system for screening and detecting infectious diseases.

### DETAILED DESCRIPTION

The following description is of exemplary embodiments and methods of use that are presently contemplated for carrying out the present invention. This description is not to be taken in a limiting sense, but is made merely for the purpose of describing the general principles and features of various aspects of the present invention. The scope of the present invention is not limited by this description.

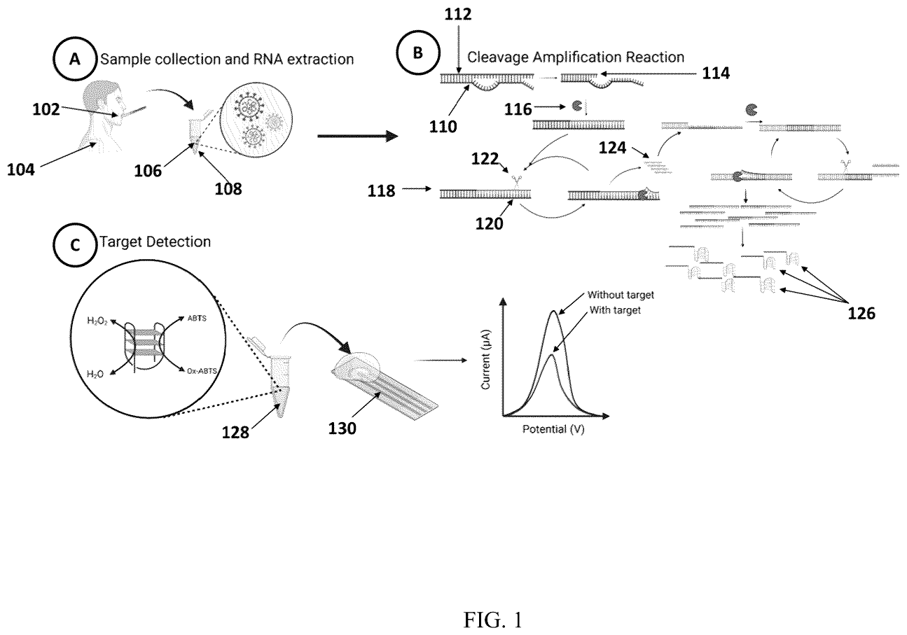

FIG. 1 shows an exemplary method and process for screening and detecting infectious diseases. The method comprises collecting an RNA/DNA sample 102 from a subject 104. It is contemplated that the sample 102 may be collected using any suitable means, including but not limited to, an oral swab, a nasal swab, a cervical swab, a blood collecting swab, or any other suitable means for collecting RNA/DNA from the subject 104. It is further contemplated that the sample 102 may be collected using any suitable instrument, including but not limited to, a cotton swab or any other suitable instrument for collecting RNA/DNA from the subject 104.

The sample 102 may then be introduced to a reagent solution 106 to form a reaction mixture 108. In some embodiments, the sample 102 may be treated with a buffer to elute and stabilize the RNA/DNA prior to introducing the sample 102 to the reagent solution 106.

The reagent solution 106 can include at least one DNAzyme 110. The DNAzyme 110 can be a single-stranded nucleic acid that can be obtained or designed through in-vitro selection.

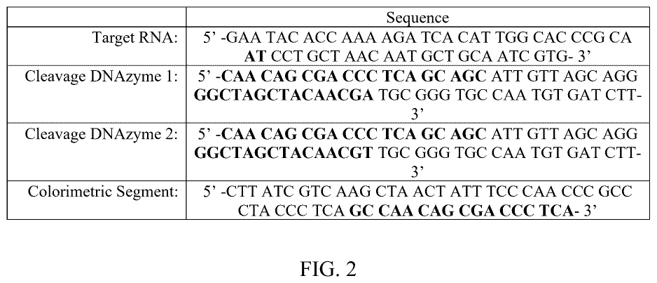

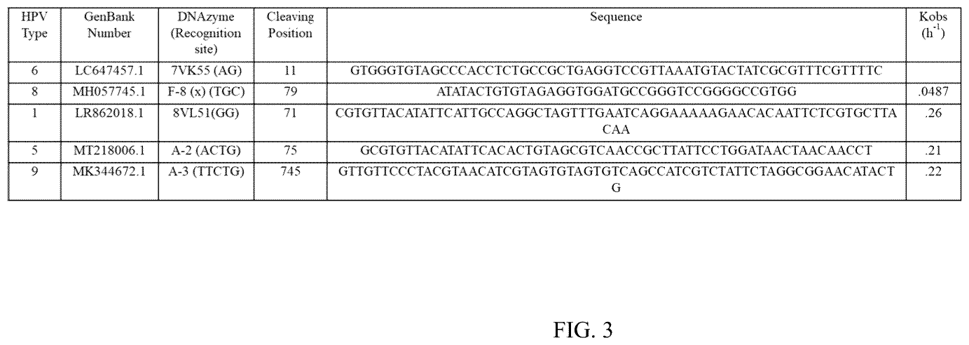

The DNAzyme 110 can be used to compliment and target a specific RNA/DNA sequence 112. DNAzyme 110 can include RNA/DNA sequences designed to target specific infectious diseases, including but not limited to, SARS-CoV-2, HPV, valley fever, hepatitis C (HCV), syphilis, chlamydia, and gonorrhea. FIGS. 2 and 3 show tables of designed DNAzyme sequences for targeting SARS-CoV-2 and HR-HPV, respectively.

Without wishing to be bound by theory, it is contemplated that the DNAzyme 110 triggers a cleavage and amplification reaction cycle that leads to the generation of a significant amount of horseradish peroxidase-mimicking DNAzyme 126. More specifically, it is contemplated that the DNAzyme 110 includes segments that are complimentary to a target RNA/DNA sequence 112 and which are used to bind to the RNA/DNA. It is understood that the target RNA/DNA sequence 112 relates to an expression profile linked to at least one infectious disease of interest. The DNAzyme 110 further includes a catalytic core that can cleave the RNA/DNA sequence 112, thus resulting in site-specific cleavage of the target RNA/DNA sequence 112 by the DNAzyme 110. The cleaved RNA/DNA 114 serves as a primer for DNA polymerase 116 to replicate the sequence of the DNAzyme 110 and yield a double stranded nucleic acid 118. The double stranded nucleic acid 118 contains a recognition site 120 for a nicking enzyme 122 such that cleavage of the double stranded nucleic acid 118 by the nicking enzyme 122 generates a new site for extension carried out by the DNA polymerase 116. Extension necessarily results in another recognition site for the nicking enzyme, and the extension/cleavage process can be repeated continuously in cycles such that a large amount of short RNA/DNA sequences 124 and ultimately horseradish peroxidase-mimicking DNAzyme 126 are generated. The horseradish peroxidase-mimicking DNAzyme 126 serves as a signal that the infectious disease is present in the RNA/DNA of the subject 104.

The cleavage and amplification reaction cycle is isothermal and may take place at low temperatures (e.g., 37° C.) in comparison to other amplification techniques, such as RT-LAMP, and thus does not require any specialized equipment (e.g., incubators).

In order to detect the presence of an infectious disease in the subject 104, an oxidizing solution (100 μL) comprising hemin, at least one organic substrate, and hydrogen peroxide may be added to the reaction mixture 108 to form a detection mixture 128. In exemplary embodiments, the organic substrate may be selected from the group consisting of ABTS, OPD, AmplexRed, DAB, AEC, TMB, homovanillic acid, luminol, or any other suitable organic substrate and mixtures thereof. In other embodiments, the DNAzyme may include ABTS, OPD, AmplexRed, DAB, AEC, TMB, homovanillic acid, luminol, or any other suitable organic substrate and mixtures thereof. It is contemplated that the significant amount of horseradish peroxidase-mimicking DNAzyme 126 present in the reaction mixture 108 catalyzes the oxidation of the at least organic substrate in the presence of hydrogen peroxide. This oxidation event serves as a signal that the infectious disease is present in the RNA/DNA of the subject 104.

In exemplary embodiments, the detection mixture 128 may then be deposited on the surface of an electrochemical sensor 130 such that CV data may be recorded, preferably immediately. The oxidation event described above may be detected through the electrochemical sensor, thus indicating the presence of an infectious disease in the subject 104.

In alternative embodiments, the detection mixture 128 may be utilized for colorimetric sensing. For example, instead of using an electrochemical sensor 130 to assess the occurrence of amplification, the color change of the detection mixture 128 may be observed, as the color change occurs due to the oxidation of the at least organic substrate by the horseradish peroxidase-mimicking DNAzyme 126 in the presence of hydrogen peroxide. Accordingly, a color change would indicate the presence of an infectious disease in the subject 104.

In alternative embodiments, the detection mixture 128 may be utilized for lateral flow detection.

Embodiments further relate to an electrochemical genosensor test that may be used for the screening and detection of infectious diseases.

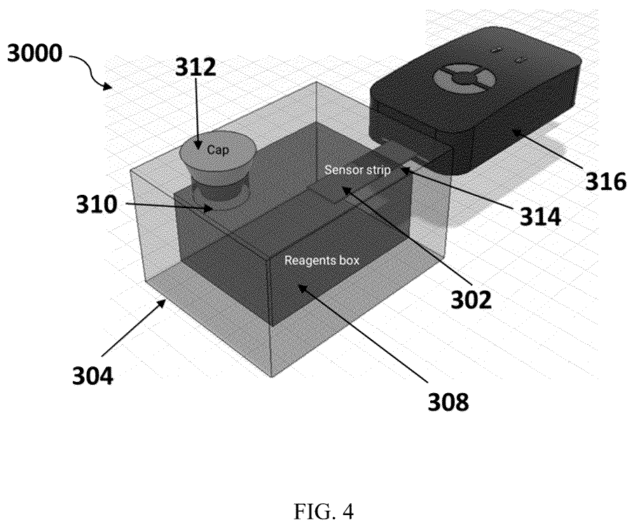

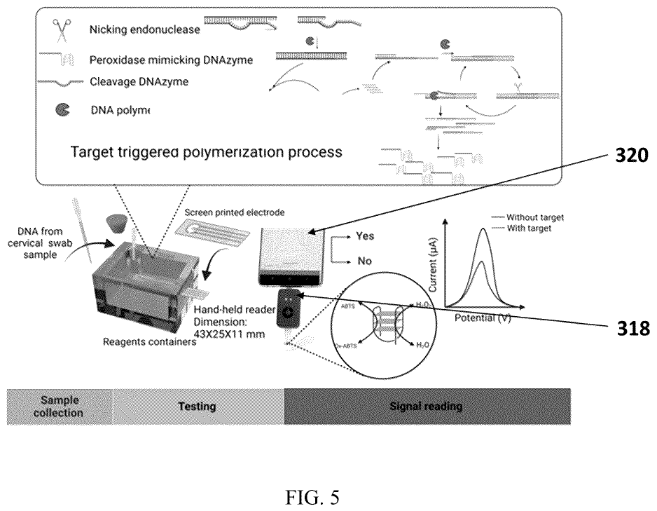

FIGS. 4-5 show exemplary electrochemical genosensors configured to carry out embodiments of the method and process (see FIG. 1) described above. The genosensor 3000 can include a sensor strip 302 and a reagent box 304.

It is contemplated that the sensor strip 302 can be an electrochemical sensor. In exemplary embodiments, the sensor strip 302 comprises electrodes 306. The electrodes 306 may be electrically connected to each other. The electrodes 306 can include a working electrode, a reference electrode, and a counter electrode. The working electrode can be configured to monitor the oxidation or reduction of a solution in contact with or near the surface of the electrode. The reference electrode can be configured to provide a stable potential for controlled regulation of the working electrode potential and allow the measurement of the potential of the working electrode without passing current through the reference electrode. The counter electrode (or auxiliary electrode) can be configured to establish a connection to a solution such that a current may be applied to the working electrode.

The electrodes 306 can be configured to perform electro-oxidation of a detection mixture and to convert an electrochemical reaction into a measurable electrical signal to effectively screen for and detect an infectious disease. For example, with respect to the method and process as described above, a significant amount of horseradish peroxidase-mimicking DNAzyme present in a reaction mixture can catalyze the oxidation of at least organic substrate in the presence of hydrogen peroxide at the working electrode, thus leading a current flow with a magnitude proportional to the horseradish peroxidase-mimicking DNAzyme concentration that can be detected by the one or more electrodes 306.

It is contemplated that that electrodes 306 can be screen printed electrodes wherein an ink is printed on a substrate. It is contemplated that the ink may be based on any suitable metal or metal oxide. In a preferred embodiment, the ink of the working electrode comprises gold, copper, silver, platinum, or other suitable material.

In exemplary embodiments, the reagent box 304 can include a reagent cavity 308, wherein the reagent cavity 308 is a hollowed-out portion (e.g., an empty space enclosed within the reagent box 304) of the reagent box 304. The reagent box 304 may be any three-dimensional shape such that the reagent box 304 may support the reagent cavity 308. The reagent box 304 can be considered a reagent housing or other type of reagent container.

The reagent box 304 can include a reagent solution (e.g., reagent solution 106, as described above). It is contemplated that the reagent solution may be positioned within the reagent cavity 308 of the reagent box 304. The reagent solution can be stored in a vessel within the reagent box or other type of reagent solution containing element of the reagent box 304.

In exemplary embodiments, spent reagent solution may be replaced with fresh (e.g., not yet used) reagent solution after use. In other words, the reagent solution can be replaceable so that the box 304 can be utilized for multiple samples or numerous different tests. For example, the reagent box 304 may be cleaned with deionized (DI) water after use such that the genosensor 3000 may be reused for another test.

For instance, the reagent box 304 can have an opening 310 size and configured to facilitate the addition and/or removal of solutions to/from the reagent cavity 308. The reagent box 304 can also have a cap 312 to effectively close the opening 310 and seal the reagent box 304 such that mixtures/solutions may no longer be added to and/or removed from the reagent cavity 308 via the opening 310 when the cap is positioned to close the opening 310. The cap 312 can be removable from the reagent box to permit emptying of the reagent cavity 308, refilling of that cavity and/or otherwise accessing the cavity 308 when in an open position and closing off that cavity 308 when in the closed position.

In an exemplary use, an RNA/DNA sample may be collected from a subject as noted above (e.g., an animal, a human, etc.). The sample may then be introduced to the reagent box 304 and be mixed with the reagent solution to form a reaction mixture. In some embodiments, the sample may be treated with a buffer to elute and stabilize the sample prior to being introduced to the reagent box 304 and being exposed to the reagent solution.

Additionally, an oxidizing solution may be introduced to the reagent box 304 for use in forming a detection mixture. The oxidizing solution can be maintained in a vessel of the box 304 and a portion of the oxidizing solution stored in the box can be subsequently mixed with the reaction mixture formed from the sample within the box 304 and the reagent solution stored in the box (e.g. via an injector and/or mixing mechanism) for testing of the sample and detection of a disease condition (e.g. whether the sample indicates a patient has HPV, etc.).

The reagent box 304 may further comprise a sensor strip inlet 314, wherein a first end of the sensor strip 302 is configured to be inserted into the sensor strip inlet 314. It is contemplated that the sensor strip inlet 314 can be sized to complement the cross-sectional shape of the first end of the sensor strip 302 for receipt of the sensor strip 302 via the sensor strip inlet 314.

In exemplary embodiments, the genosensor 3000 can also include a mobile reader 316. In some embodiments, the mobile reader 316 can be a hand-held reader. For example, the mobile reader 316 can be configured as a mobile potentiostat.

A second end of the sensor strip 302 can be configured to be inserted into the reader 316 as (or after) the first end of the sensor strip 302 is inserted into the sensor strip inlet 314 of the box 304. The reader 316 can be an electronic instrument that controls the voltage difference between the working electrode and the reference electrode by injecting current through the counter electrode. The reader 316 can measure the current flow between the working electrode and the counter electrode. It is further understood that the controlled variable in the reader 316 can be cell potential and the measured variable can be cell current. Accordingly, the reader 316 can be configured to collect and record CV data via the sensor strip 302.

For example, in an exemplary use, as the first end of the sensor strip 302 is inserted into the sensor strip opening 314, it is contemplated that a detection mixture 128 may be deposited on the sensor strip 302 (e.g., a mixture of the sample, reaction mixture 108 and oxidizing solution can be deposited on strip 302 within the box 304). An oxidation event may be detected by the sensor strip 302/reader 316 via the deposited detection mixture 128, thus indicating the presence of an infectious disease. For example, a sufficient electrical current or voltage that exceeds or meets a pre-selected disease identification threshold can be detected to indicate the presence of an infection. If that threshold is reached, a detection can occur. If that threshold is not met, then the sample may be found as not having the disease.

Such a detection of a disease condition or a non-disease condition can be made via the reader 316 or via an external device 320 that can receive measurement data from the reader 316 and analyze the data to determine whether the disease condition is present. The results of the data analysis from the sample can be output via a display and/or printer for outputting the result of the conducted testing.

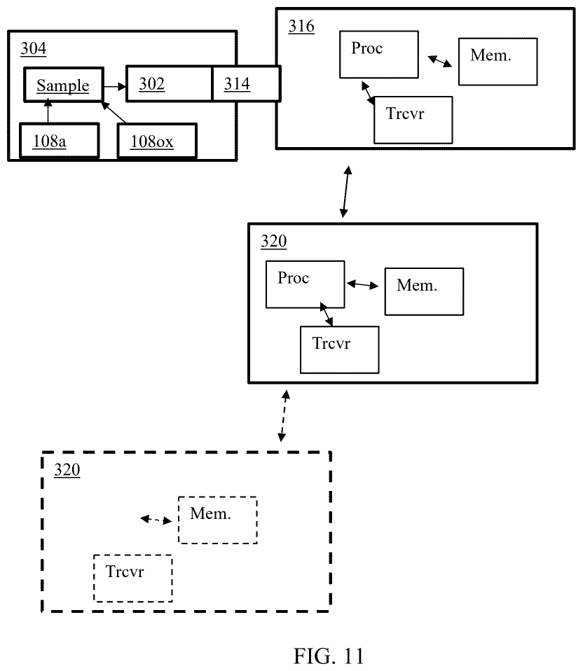

In some embodiments, there can be a first external device 320 that can be configured as a site-based computer device and a second external device 320 (shown in broken line in FIG. 11) that can be communicatively connectable to the first external device via a network connection (e.g., internet connection, wide area network connection, etc.). The second external device 320 can be configured as a host device and receive the reader data from the first external device 320 for storage and analysis of the data. The second external device can communicate with the first external device to provide data concerning the results of the analysis and other data concerning the collected reader data for display to a user via the first external device 320.

In some implementations, the second external device 320 can be configured as a server or cloud based service providing device for analysis and storage of the reader data obtained via the reader 316 that can be communicated to a user via the user's first external device 320, which can be a tablet, smart phone, laptop computer, personal computer, or other type of terminal device that can be used to utilize services of the second external device. Such services can be effectuated via an application programming interface (API) and/or use of an application stored on the first external device 320 that is supported via the second external device 320.

It is contemplated that the reader 316 may be portable. The portability of the reader 316 can provide numerous advantages, including but not limited to, allowing the genosensor 3000 to be used as a point-of-care test.

The reader 316 can include an external power source (e.g., the reader 316 may be USB-powered) and/or an internal power source (e.g., the reader 316 may be powered by a battery positioned within the reader 316).

The reader 316 can include a connector element 318. The connector element 318 may be any suitable connector element, such as a USB (e.g., types A, B, C) port, a USB mini (e.g., types A, B, C) port, a USB micro (e.g., types A, B, C) port, a lightning port, or any other suitable connector element. In embodiments wherein the reader 316 comprises an external power source, the connector element 318 may be configured to connect the reader 316 to the external power source. The connector element 318 may further be configured to connect the reader 316 to an external device 320 (e.g., smartphone, app, watch (e.g., smart watch), computer, etc.), such that information collected by the reader 316, such as CV data, may be stored, analyzed, and/or displayed using the external device 320. It is contemplated that the external device 320 may serve as the external power source.

The reader 316 can be a type of machine that can include a processor (Proc) connected to a non-transitory memory (Mem.) and at least one transceiver (Trcvr) for forming communicative connections with one or more other devices. The at least one transceiver (Trcvr) can include a Bluetooth module and/or other type of transceiver unit (Trcvr) such that the reader 316 may be configured for use with the external device 320 (e.g., smartphone, app, watch (e.g., smart watch), laptop computer, desktop computer, etc.), such that information collected by the reader 316, such as CV data, may be transmitted to the external device 320 so that data can be stored, analyzed, and/or displayed using the external device 320. Also, or alternatively, the reader 316 can include a wireless local network transceiver (e.g., a Wi-Fi transceiver unit) or other type of wireless communication module so that information collected by the reader 316, such as CV data, may be communicated to the external device 320 so it can be stored, analyzed, and/or displayed using the external device 320.

The processor can be hardware (e.g., processor, integrated circuit, central processing unit, microprocessor, core processor, computer device, etc.), configured to perform operations by execution of instructions embodied in algorithms, data processing program logic, artificial intelligence programming, automated reasoning programming, etc. that can be defined by code stored in the memory. The processor can facilitate receipt, processing, and/or storage of readings from at least one sensor of the reader 316 and/or control transmission of the collected data to the external device 320.

It should be noted that use of processors herein can include any one or combination of a Graphics Processing Unit (GPU), a Field Programmable Gate Array (FPGA), a Central Processing Unit (CPU), etc. The processor can include one or more processing or operating modules. A processing or operating module can be a software or firmware operating module configured to implement any of the functions disclosed herein. The processing or operating module can be embodied as software and stored in memory, the memory being operatively associated with the processor. A processing module can be embodied as a web application, a desktop application, a console application, etc.

The memory (Mem.) can be a non-transitory computer readable memory configured to store data. Embodiments of the memory can include a processor module and other circuitry to allow for the transfer of data to and from the memory, which can include to and from other components of a communication system. This transfer can be via hardwired links or wireless transmission communication links. The communication system can include transceivers, which can be used in combination with switches, receivers, transmitters, routers, gateways, wave-guides, etc. to facilitate communications between different devices via a communication approach or protocol for controlled and coordinated signal transmission and processing to any other component or combination of components of the communication system. The transmission can be via a communication link, which can be a wireless type of communication connection and/or a wired type of connection.

The computer or machine-readable medium can be configured to store one or more instructions thereon. The instructions can be in the form of algorithms, program logic, etc. that cause the processor to execute any of the functions disclosed herein.

The processor can be in communication with other processors of other devices (e.g., a second external device, a computer system, a laptop computer, a desktop computer, etc.). An exemplary other device can be a Bluetooth enabled device, near field communication device, etc. Any of those other devices can include any of the exemplary processors disclosed herein as well as transceivers or other communication devices/circuitry to facilitate transmission and reception of wireless signals or other type of communicative connections.

The reader 316 can also include other elements, such as input devices (e.g., microphone, keyboard, keypad, touch screen, pointer device, etc.) and output devices (e.g., displays, speakers) communicatively connectable to the processor. The input devices and output devices can be provided and arranged to permit a user to provide input to the reader 316 to control operation of the reader and receive output from the reader 316 (e.g., via speaker and/or display).

As can be appreciated from the above, each external device 320 can also include at least one processor (Proc) communicatively connected to a non-transitory memory (Mem.) and at least one transceiver (Trcvr). The external device can be communicatively connectable to the reader 316 to receive data from the reader collected via the box 304 and sample for storage and analysis. The external device 320 can be configured to analyze the data collected via the reader to detect whether the sample indicated a subject, or patient, had a disease, for example. The external device 320 can also include other elements, such as input devices (e.g., microphone, keyboard, keypad, touch screen, pointer device, etc.) and output devices (e.g., displays, speakers) communicatively connectable to the processor.

### EXAMPLES

### Example 1

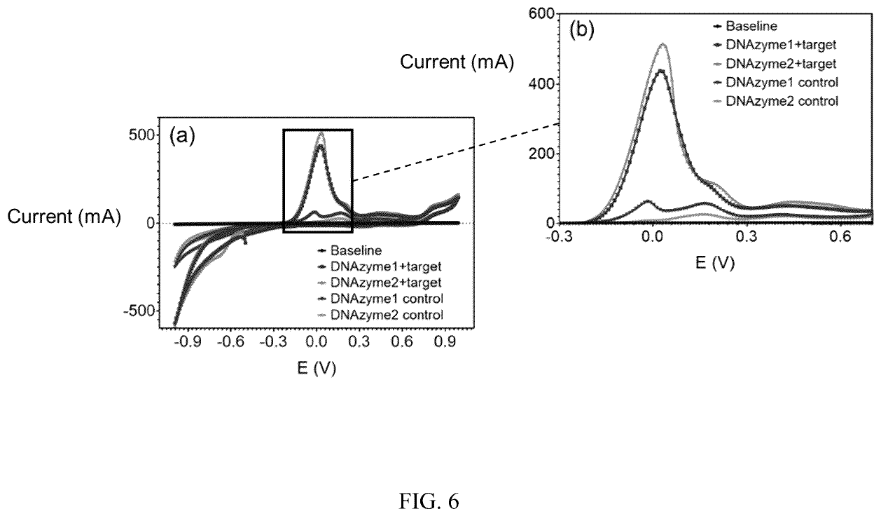

As seen in FIG. 2, two DNAzyme sequences (SEQ ID NO. 2 and SEQ ID NO. 3) were designed to target the SARS-CoV-2 virus (SEQ ID NO. 1). A colorimetric segment (SEQ ID NO. 4) was also provided. The DNAzyme sequences were then tested to evaluate their performance. FIG. 6 shows that DNAzyme 2 (SEQ ID NO. 3) provided for the highest current value in the presence of the target (SEQ ID NO. 1) and showed the lowest signal in the absence of the target.

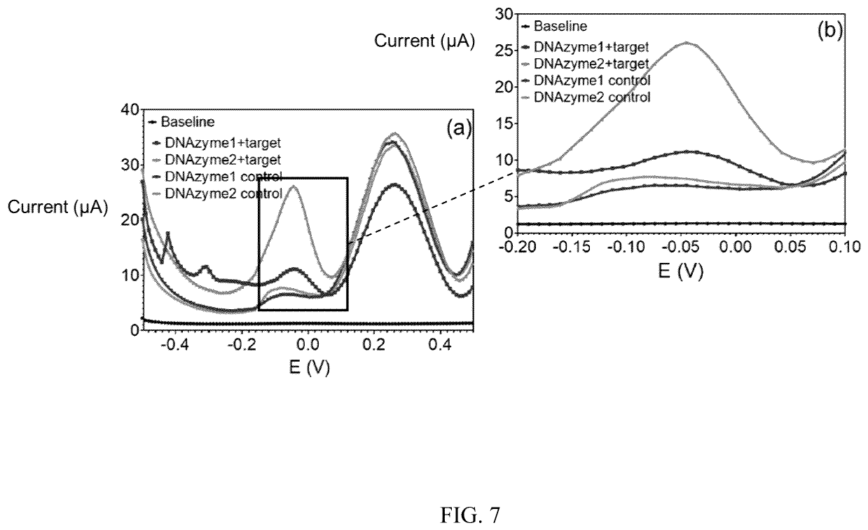

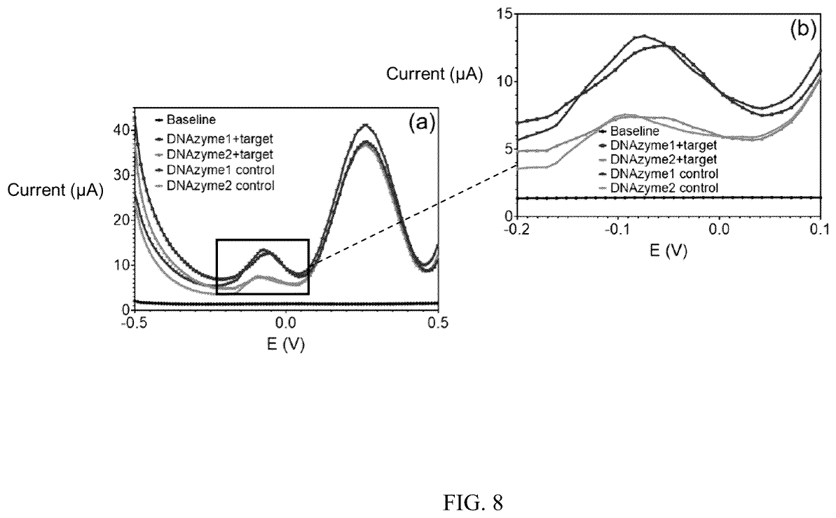

The electrochemical measurement used to detect the target was also optimized. The response of CV, DPV, and SWV were evaluated, as shown in FIGS. 6-8, respectively. It was found that CV measurements provided the best discrimination between the target and the control.

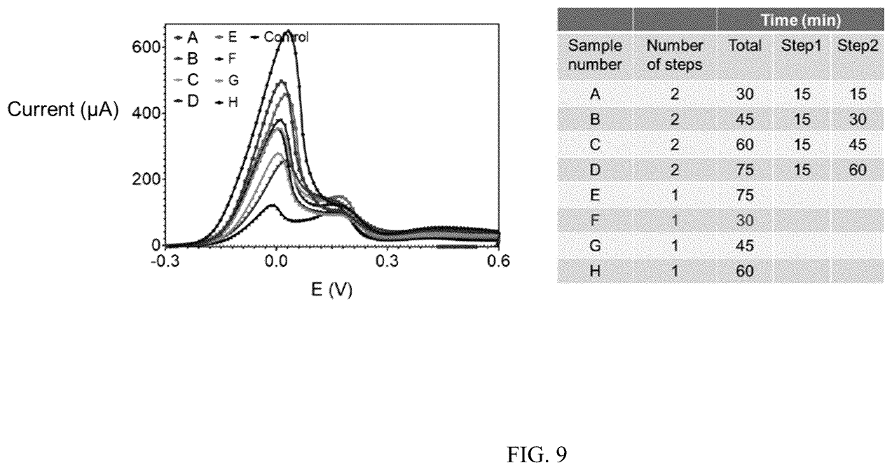

The DNAzyme process takes approximately 3 hours in the conducted experimentation, and it was carried out in a two-step reaction consistent with the process discussed above in conjunction with FIG. 1. As summarized in FIG. 9, decreasing the time and number of steps to simplify the test was investigated in an attempt to simplify the test and make it more easily applicable. The number of steps was reduced from two steps (e.g., approximately 3 hours) to a one-step reaction (e.g., approximately 30 minutes). Results from our experiments confirmed a highly accurate quantitative test for nucleic acid detection of SARS-CoV-2.

### Example 2

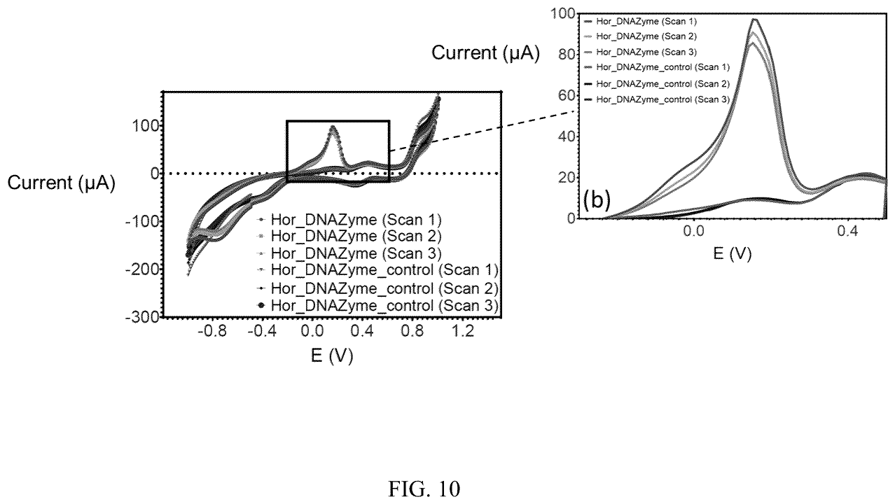

As seen in FIG. 3, five DNAzyme sequences (SEQ ID NO. 5, SEQ ID NO. 6, SEQ ID NO. 7, SEQ ID NO. 8, and SEQ ID NO. 9) were designed to target various types of HR-HPV. The DNAzyme sequences were then tested to evaluate their performance, as seen in FIG. 10.

Results from our experiments confirmed a highly accurate quantitative test for nucleic acid detection of HPV with a sample-to-assay time of 30 minutes. FIG. 10 illustrates representative electrochemical readouts as a response to the presence of the target as compared to the negative samples. The DNAzyme-triggered polymerization process showed a significant current peak in the presence of the target. However, in the absence of the target negligible current was observed.

It should be understood that modifications to the embodiments disclosed herein can be made to meet a particular set of design criteria. For instance, the number of or configuration of components or parameters may be used to meet a particular objective.

It will be apparent to those skilled in the art that numerous modifications and variations of the described examples and embodiments are possible in light of the above teachings of the disclosure. The disclosed examples and embodiments are presented for purposes of illustration only. Other alternative embodiments may include some or all of the features of the various embodiments disclosed herein. For instance, it is contemplated that a particular feature described, either individually or as part of an embodiment, can be combined with other individually described features, or parts of other embodiments. The elements and acts of the various embodiments described herein can therefore be combined to provide further embodiments.

It is the intent to cover all such modifications and alternative embodiments as may come within the true scope of this invention, which is to be given the full breadth thereof. Additionally, the disclosure of a range of values is a disclosure of every numerical value within that range, including the end points. Thus, while certain exemplary embodiments of the apparatus and process and/or utilization and methods of making and using the same have been discussed and illustrated herein, it is to be distinctly understood that the invention is not limited thereto but may be otherwise variously embodied and practiced within the scope of the following claims.

What is claimed is:

1. A method for detecting an infectious disease, comprising:
introducing a sample comprising RNA and/or DNA collected from a subject to a reagent solution to form a reaction mixture, wherein the reagent solution comprises at least one DNAzyme configured to target and amplify an RNA and/or DNA expression profile linked to a pre-selected infectious disease; and
adding an oxidizing solution to the reaction mixture to form a detection mixture, wherein the oxidizing solution comprises at least one organic substrate and hydrogen peroxide.

2. The method of claim 1, wherein the reaction mixture comprises a horseradish peroxidase-mimicking DNAzyme to catalyze an oxidation reaction of the at least one organic substrate with the sample.

3. The method of claim 2, further comprising:
depositing the detection mixture on a surface of a sensor strip, wherein the sensor strip comprises at least one electrode configured to detect the oxidation reaction.

4. The method of claim 3, further comprising:
integrating the sensor strip with a reader configured to collect and record data for detecting the oxidation reaction.

5. The method of claim 4, further comprising:
integrating the reader with an external device configured to display and/or store and/or analyze the data for detecting the oxidation reaction.

6. The method of claim 1, wherein the infectious disease is selected from the group consisting of SARS-CoV-2, HPV, HCV, syphilis, chlamydia, and gonorrhea.

7. The method of claim 1, wherein the oxidizing solution further comprises hemin.

8. The method of claim 1, the method further comprising:
treating the sample with a buffer to stabilize the RNA and/or DNA prior to introducing the sample to the reagent solution.

9. The method of claim 1, wherein the at least one organic substrate is selected from the group consisting of ABTS, OPD, AmplexRed, DAB, AEC, TMB, homovanillic acid, luminol, and mixtures thereof.

10. The method of claim 2, further comprising:
detecting the oxidation reaction via colorimetric sensing or via lateral flow detection.

11. The method of claim 2, further comprising:
interpreting the detection of the oxidation reaction as an indication of the presence of the pre-selected disease and non-detection as an indication of its absence.

12. An apparatus for detecting an infectious disease, comprising:
a genosensor, wherein the genosensor comprises:
a housing having (a) a reagent solution retaining vessel having a reagent solution comprising at least one DNAzyme configured to target and amplify an RNA and/or DNA expression profile for a pre-selected infectious disease and (b) an oxidizing solution retaining vessel having an oxidizing solution comprising at least one organic substrate and hydrogen peroxide;
the housing configured to receive a sample having RNA and/or DNA collected from a subject for mixing with the reagent solution and the oxidizing solution for depositing on a sensor strip positionable within the reagent housing.

13. The apparatus of claim 12, further comprising:
a reader connectable to the sensor strip to detect an oxidation reaction from a mixture of the sample, reagent solution, and oxidizing solution deposited on the sensor strip, and
at least one external device communicatively connectable to the reader to receive data collected by the reader to determine whether the oxidation reaction was detected to indicate the sample had the pre-selected infectious disease.

14. The apparatus of claim 12, wherein the housing is configured so that the sample, the reagent solution and the oxidizing solution are mixable to form a detection mixture that is depositable on the detection strip.

15. The apparatus of claim 14, wherein the sample, the reagent solution and the oxidizing solution are mixable at the same time via a mixing mechanism within the housing to form the detection mixture for depositing on the detection strip.

16. The apparatus of claim 12, wherein the housing is configured such that the sample and the reagent solution are mixable to form a reaction mixture and the oxidizing solution is mixable with the reaction mixture to form a detection mixture that is depositable on the detection strip via a mixing mechanism within the housing.

17. A method for detecting an infectious disease, comprising:
mixing a sample comprising RNA and/or DNA collected from a subject with a reagent solution and an oxidizing solution to form a detection mixture, wherein the reagent solution comprises at least one DNAzyme, wherein the DNAzyme is configured to target and amplify an RNA and/or DNA expression profile linked to a pre-selected infectious disease; and
determining whether an oxidation reaction occurs within the detection mixture to determine whether the pre-selected infectious disease is present in the sample such that a detection of the oxidation reaction indicates the pre-selected infectious disease is present and a non-detection of the oxidation reaction indicates the pre-selected infectious disease is not present.

18. The method of claim 17, wherein the reaction mixture comprises a horseradish peroxidase-mimicking DNAzyme, and wherein the at horseradish peroxidase-mimicking DNAzyme is configured to catalyze an oxidation reaction of the at least one organic substrate with the sample.

19. The method of claim 18, further comprising:
depositing the detection mixture on a surface of a sensor strip, wherein the sensor strip comprises at least one electrode configured to detect the oxidation reaction.

20. The method of claim 19, further comprising:
connecting the sensor strip with a reader configured to collect and record data for detecting the oxidation reaction.

21. The method of claim 20, further comprising:
communicating the data for detecting the oxidation reaction to at least one external device for analysis of the data to determine whether the pre-selected infectious disease is present in the sample.

22. The method of claim 17, wherein the detection of the oxidation reaction occurs via colorimetric sensing or via lateral flow detection.


SEQUENCE LISTING (9 sequences)

SEQ ID NO: 1 - RNA, 58 nt, synthetic construct
  source 1..58: mol_type = other RNA; note = target RNA of SARS-CoV-2
gaatacaccaaaagatcacattggcacccgcaatcctgctaacaatgctgcaatcgtg

SEQ ID NO: 2 - DNA, 68 bp, synthetic construct
  source 1..68: mol_type = other DNA; note = cleavage DNAzyme
caacagcgaccctcagcagcattgttagcaggggctagctacaacgatgcgggtgccaatgtgatctt

SEQ ID NO: 3 - DNA, 68 bp, synthetic construct
  source 1..68: mol_type = other DNA; note = cleavage DNAzyme
caacagcgaccctcagcagcattgttagcaggggctagctacaacgttgcgggtgccaatgtgatctt

SEQ ID NO: 4 - DNA, 68 bp, synthetic construct; identical to SEQ ID NO: 3
  source 1..68: mol_type = other DNA; note = colorimetric segment

SEQ ID NO: 5 - DNA, 60 bp, synthetic construct
  source 1..60: mol_type = other DNA
  misc_feature 11: note = cleaving position
gtgggtgtagcccacctctgccgctgaggtccgttaaatgtactatcgcgtttcgttttc

SEQ ID NO: 6 - DNA, 40 bp, synthetic construct
  source 1..40: mol_type = other DNA
atatactgtgtagaggtggatgccgggtccggggccgtgg

SEQ ID NO: 7 - DNA, 66 bp, synthetic construct
  source 1..66: mol_type = other DNA
cgtgttacatattcattgccaggctagtttgaatcaggaaaaagaacacaattctcgtgcttacaa

SEQ ID NO: 8 - DNA, 58 bp, synthetic construct
  source 1..58: mol_type = other DNA
gcgtgttacatattcacactgtagcgtcaaccgcttattcctggataactaacaacct

SEQ ID NO: 9 - DNA, 59 bp, synthetic construct
  source 1..59: mol_type = other DNA
gttgttccctacgtaacatcgtagtgtcagccatcgtctattctaggcggaacatactg